(19) United States
(12) Patent Application Publication
(10) Pub. No.: US 2015/0105780 A1
(43) Pub. Date: Apr. 16, 2015

(54) METHOD AND SYSTEM FOR DETERMINING THE ALIGNMENT OF TWO BONES

(71) Applicant: OSTESYS, Plouzane (FR)

(72) Inventors: Guillaume Dardenne, Plouzane (FR); Stéphane Lavallee, St. Martin D'uriage (FR)

(73) Assignee: OSTESYS, Plouzane (FR)

(21) Appl. No.: 14/381,236

(22) PCT Filed: Feb. 25, 2013
(86) PCT No.: PCT/EP2013/053706

§ 371 Date: Aug. 27, 2014

Related U.S. Application Data
(60) Provisional application No. 61/605,203, filed on Mar. 1, 2012.

(30) Foreign Application Priority Data
Mar. 27, 2012 (FR) 1252701

(51) Int. Cl. A61B 17/17

(52) U.S. Cl. CPC A61B 17/17
USPC 606/87

(57) ABSTRACT

The present invention relates in particular to a method of alignment of a first bone, with a first and a second ends, in relation with a second bone, with a first and a second end, both bones being in a same limb, the first end of said first bone being a part of a distal joint for the first bone, the second end of said second bone being a part of a proximal joint for the second bone, said second end of the first bone and said first end of the second bone constituting a middle joint between both bones, wherein this first bone will be partially cut, in a transversal way, into two segments to form a hinge and wherein a tracker has been attached beforehand to each of two said bone segments.

The present invention relates to a method and a system for determining the alignment of two bones.

Osteoarthritis is a mechanical abnormality involving degradation of the joints, including articular cartilage and subchondral bone.

This pathology can be treated using different methods. The most common one is the total replacement of the diseased joint with a prosthetic implant. This method is however very degenerative, invasive and sometimes traumatic for the patient.

When this disease is the consequence of a misalignment of a limb, it can be delayed or treated with an osteotomy. This surgical procedure aims to rebalance the stress on the diseased joint by realigning the joint centers of the limb (hip, knee and ankle centers for the lower limb and shoulder, elbow and wrist for the upper limb). A partial cut is thus performed on one of the two bones forming the limb (femur or tibia for the lower limb, and, humerus or radius for the upper limb). The deformation is made by rotating the cut bones around a rotation point called hinge resulting from the partial cut. Once the alignment has been reached, the two cut bony segments are maintained in the wanted position, in most cases, with an osteosynthesis implant.

Despite its efficiency, this surgical procedure is however very difficult to perform because it requires a high accuracy in order to obtain good long-terms results. This accuracy is however very difficult to reach for several reasons
the plan of the surgery is made in two dimensions (2D) even though the surgical procedure must be done in three dimensions (3D),
the planned surgery is extremely difficult to perform because the correction angle is planned pre-operatively and is transferred intra-operatively on the patient with inaccurate geometric tools like protractors or rules),

Moreover, x-ray images with C-Arm are systematically realized intraoperatively in order to check and control the gesture.

All these reasons make the procedure inaccurate and expose the clinical staff and the patient to radiations.

In this context, several solutions based on computer assisted surgery systems have been developed in order to improve the accuracy, the reliability of this surgical procedure and avoid the use of X-ray images. These systems can be used intra-operatively and allows the surgeon to obtain 3D information about the alignment. Most of them are composed of a 3D optical localizer which can monitor and detect markers in the space. A computer connected to this localizer can therefore obtain the 3D position and orientation of the different trackers composed of, at least, three markers. These trackers are fixed to the bones allowing the system to know the 3D position and orientation of the bones in the space.

To obtain the 3D alignment of a limb, the three joint centers which compose this limb must be detected and recorded with the navigation system, for instance, the hip, the knee and the ankle centers for the lower limb.

All the existing solutions require, at least, two trackers fixed on each bone in order to obtain their 3D positions and orientations: for instance one tracker on the femur and one tracker on the tibia for the lower limb.

The joint centers are therefore estimated with respect to these trackers. For instance, the hip and the knee centers are computed with respect to the femur tracker and the ankle center is estimated with respect to the tibia tracker for the lower limb. Several methods can be used to estimate these joint centers: functional methods based on the relative motions of the bones composing the joint, morphologic methods based on the 3D acquisition of specific anatomical points, or morpho-functional methods based on functional and morphologic information.

The hip center can be obtained, for instance, with a functional method: a circumduction motion of the femur is performed around the hip. The navigation system records the 3D positions and orientations of the tracker fixed to the femur during the motion. The center of rotation of the acquired data is computed and represents the hip center. The knee joint can be estimated, via a morpho-functional method, by making a flexion-extension. 3D positions and orientations of the femur and tibia trackers are recorded during the motion by the navigation system. An average rotation axis representing the flexion/extension is estimated. The knee center is the average of two points, representing the medial and lateral condyle, acquired on this axis with a digitizer recognizable by the station. The ankle center can be estimated with a morphologic method: the middle of the medial and lateral point of the ankle.

The alignment represented by the angle between both bones, for instance, the angle HKA (Hip-Knee-Ankle) between the mechanical axis of the femur and the mechanical axis of the tibia can be computed in real time thanks to the navigation system during the deformation process.

The traditional techniques allowing a computer assisted surgery system to compute alignment information after an osteotomy are all based on, at least, two trackers: one on each bone. These traditional techniques require thus the fixation of at least one tracker outside the incision. For instance, for a tibia osteotomy, the surgical incision is performed on the anterior medial part of the tibia. The placement of the femur tracker requires thus an additional incision on the femur and an additional time.

In others situations, a unique tracker can be used to obtain the alignment information of the limb. These methods require a specific acquisition procedure to compute the joint centers with respect to this unique tracker. These methods can however not be used when an osteotomy is performed. In this case, one of the two bones is divided into two bony segments. One of these segments becomes therefore not localized by the navigation station: no tracker is linked with this segment. Thus, the computation of the alignment after the osteotomy cannot be performed since the navigation station does not know the 3D position and orientation of at least one of the joint centers associated to this segment.

U.S. 2004/0106861 relates to a method of determining the position of the articulation point of a joint.

U.S. 2012/016427 relates to an osteosynthesis device with an integrated tracker. So, it is directly localizable and permits to localize the bony segments obtained after an osteotomy. Nevertheless, it does not permit to compute the alignment of a limb.

In U.S. 2007/0118140, trackers are fixed on the pelvis, the femur, the tibia and the foot, in order to determine the position of the joint centers.

It is an object of the present invention to provide a method allowing a computer assisted surgery system to determine the alignment of two bones, with one of them which was cut into two segments, which does not require additional invasiveness and which is faster and less traumatic to the patient as compared to prior arts methods.

It is another object of the invention to provide an optimal acquisition sequence which can be used to reliably and accurately locate joint centers for the computation of the alignment of a limb in full extension.

In this regard, the invention is a method for determining the alignment of a first bone in relation with a second bone, both being in a same limb. The first bone will be further partially cut into two segments to form a hinge. The method requires the fixation of two trackers, in the surgical incision, on a same bone: one on each of the future two segments. Once the trackers are fixed, a circumduction motion is performed around the proximal extremity (for instance the hip or the shoulder center). This circumduction motion is performed in a fixed relative position, for instance in full extension, between the first bone and the second one. The system records the position of the proximal tracker during the motion and computes the center of the proximal joint with respect to this tracker by using existing methods such as for instance the least moving point or the center of rotation algorithms.

The middle joint center (for instance the knee or elbow center) is obtained by acquiring the medial and lateral point of the middle joint with a digitizer whose 3D position and orientation is known by the system and by computing the middle of both points. The distal joint center is obtained by digitizing the medial and lateral point of the distal joint (for instance the medial and lateral point of the ankle) with the same previous method, and by computing the middle of both points.

Once the three joint centers are known, following a fixed relative position and with respect to the two trackers, the osteotomy can be performed by keeping the two trackers on each segment. The surgeon can deform the two segments in order to obtain the desired alignment. The navigation system records the 3D positions and orientations of both trackers, and provides, with an adapted display unit, this information to the surgeon.

In other words, this method of alignment of a first bone, with a first and a second ends, in relation with a second bone, with a first and a second ends, both bones being in a same limb, the first end of said first bone being a part of a distal joint for the first bone, the second end of said second bone being a part of a proximal joint for the second bone, said second end of the first bone and said first end of the second bone constituting a middle joint between both bones, i.e. an articulation, wherein this first bone will be partially cut, in a transversal way, into two segments to form a hinge and wherein a tracker has been attached beforehand to each of two said bone segments, is characterized in that said method comprises the following steps:
placing the two bones in a fixed relative position;
pivoting the set of two bones around the said proximal joint and acquiring the point representing a center of rotation of said proximal joint;
acquiring the point representing a center of the said middle joint, which is the middle of the medial anatomical point and the lateral anatomical point of the said middle joint;
acquiring the point representing a center of the said distal joint, which is the middle of the medial anatomical point and the lateral anatomical point of the said distal joint;
cutting partially the said first bone into two segments to obtain said hinge;
pivoting the two segments of the said first bone around said hinge until reaching the desired alignment between the said second bone and the said two segments of the first bone, while acquiring with the system the relative positions of said three centers and;
visualizing the alignment of the three joint points in relation to the rotation of one segment with respect to the other one around said hinge.

According to other characteristics of this method:
the proximal joint center of the second bone is obtained by performing a pivoting motion around the said proximal joint, recording this motion with the navigation system, and computing the center of rotation, following an optimization criterion;
the middle joint center is computed by acquiring two points with a digitizer visible by the said navigation system;
the distal joint center is computed by acquiring two points with the said digitizer visible by the said navigation system.

The invention also relates to a system of alignment of a first bone, with a first and a second ends, in relation with a second bone, with a first and a second ends, both bones being in a same limb, the first end of said first bone being a part of a distal joint for the first bone, the second end of said second bone being a part of a proximal joint for the second bone, said second end of the first bone and said first end of the second bone constituting a middle joint, i.e. an articulation between both bones, said first bone being partially cut, in a transversal way, into two segments linked partially to form a hinge, which is characterized in that said system comprises:
a computer assisted surgery station, which includes a screen and a computer, this computer comprising a memory in which is recorded a computer program and a treatment unit adapted to execute this program;
two trackers adapted to be placed each one on one of the two segments of said first bone, the position and orientation of these trackers being detectable by said station, this system being also characterized by the fact that said computer program comprises instructions for implementing the following steps:
while each trackers has been fixed respectively to each of said segments, and while a rotation of both bones is made around said proximal end of said second bone, said two bones being maintained in a fixed relationship one to the other,
acquiring the point which represents the proximal end of said second bone, in the referential (framework) of one of the trackers;
the point which represents the center of said articulation, in the referential of one of said trackers;
the point which represents the distal end of said first bone, in the referential of one of said trackers;
displaying, on the screen of said station, of said points which represent the proximal end of said second bone, the center of said articulation and the distal end of said first bone, during a movement of said two segments of said first bone, relative to each other.

In all the present text, the word “acquiring” signifies “determining”.

According to other characteristics of this system:
said screen and/or said computer is (are) carried by one of said trackers;
said trackers are optic or magnetic.

The invention and its advantages will be described in more details below with references to the accompanying schematic drawings, which for the purpose of illustration show some non-limiting embodiments, and in which:

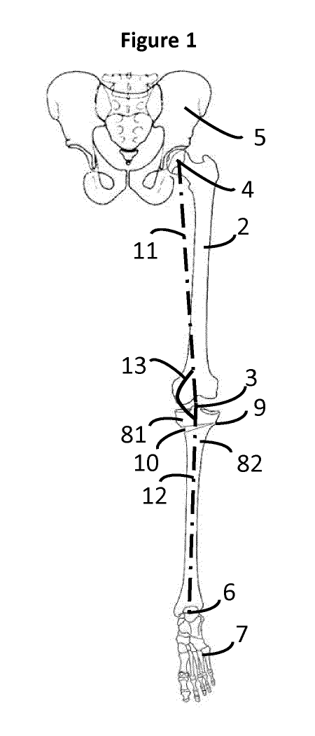

The FIG. 1 is a representation of the lower limb after osteotomy showing the alignment of the three joint centers.

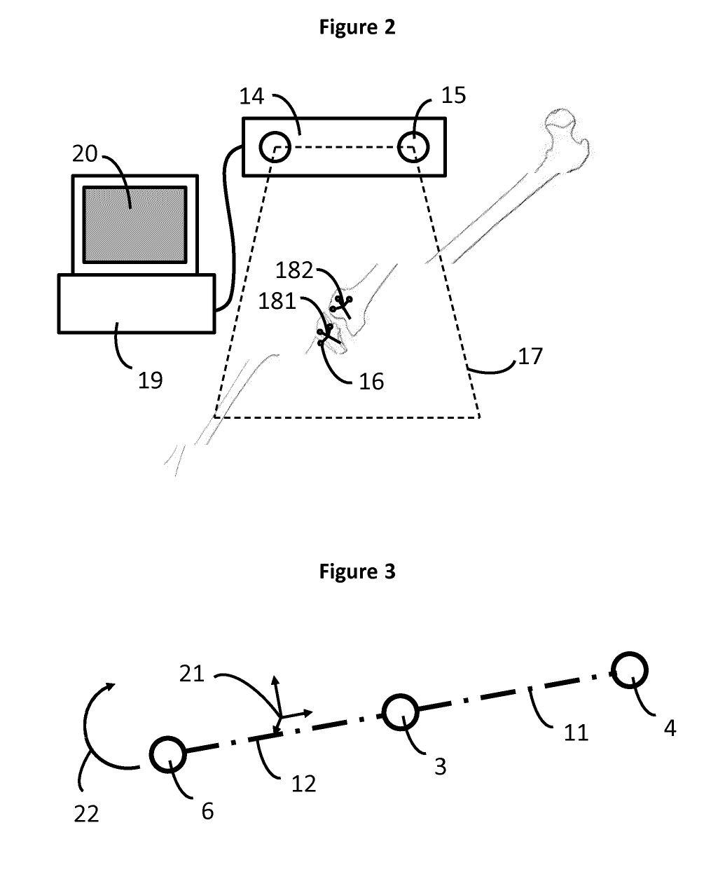

The FIG. 2 is a schematic representation of the required hardware for the determination of the 3D alignment of a limb with the method of the invention.

The FIG. 3 is a schematic drawing of the lower limb showing the acquisition of the proximal joint center.

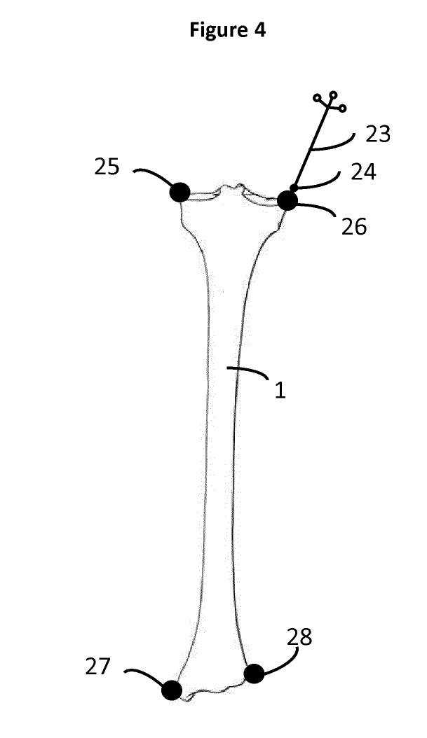

The FIG. 4 is a representation of the tibia showing the required acquisition for the lower limb for the determination of the middle joint center and the distal joint center.

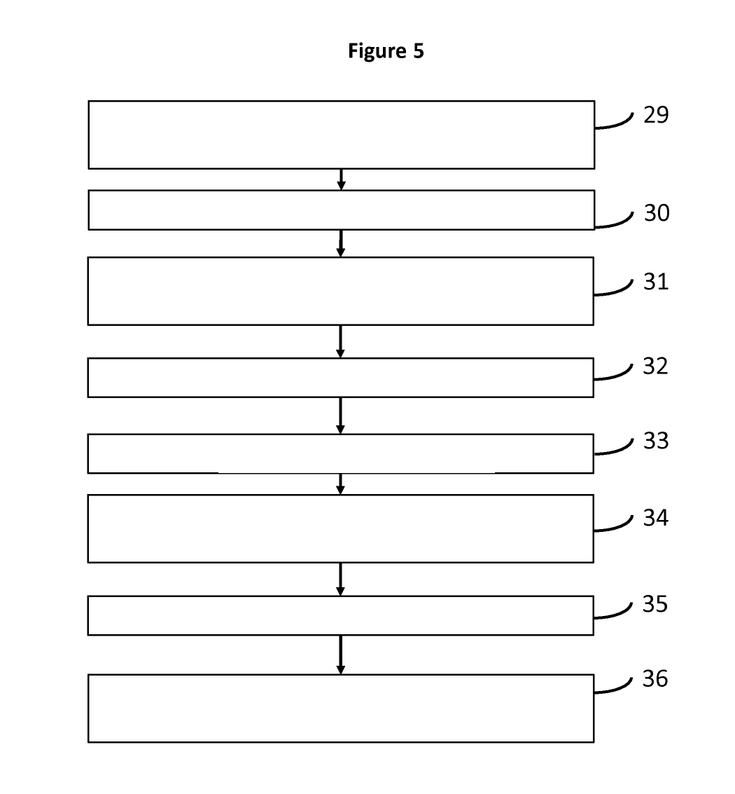

The FIG. 5 is the surgical procedure flow diagram.

The following detailed description primarily uses the lower limb as an example. However, the present invention can also be used with others limbs.

FIG. 1 and FIG. 4 shows a lower limb composed of a first bone 1 representing a tibia and a second bone 2 representing a femur. Both bones are articulated around a knee 3 called middle joint. The second bone 2 has a proximal joint 4, i.e. the hip, which makes the interface with the pelvis 5. The first bone 1 has a distal joint 6, the ankle, which makes the interface with the foot 7.

The first bone 1 is partially cut and divided into two segments 81 and 82 which represent respectively the epiphysis and the diaphysis of the tibia. These two segments 81 and 82 can pivot around the hinge 9 formed after the osteotomy 10. The mechanical axis 11 of the second bone 2 is the line passing through the proximal and the middle joints. The mechanical axis 12 of the first bone 1 is the line passing through the middle and the distal joints.

The goal of the tibia osteotomy is to correct the angle 13 between both mechanical axes. This angle 13 must be situated in the range of 183° and 186° in order to obtain good long term results.

FIG. 2 shows the required hardware for determining the limb alignment. This hardware is composed of a 3D localizer 14 equipped with cameras 15 which can determine by triangulation the 3D position of markers 16 in the space 17 visible by the localizer. Two trackers 181 and 182 of a known type, containing at least three markers 16 and allowing the computer 19 connected to the localizer 14 to compute their 3D positions and orientations, are fixed on the diaphysis and epiphysis of the tibia. The trackers 181 and 182 and the localizer 14 may be any commercially available system, such as, for instance, that marketed by the company NORTHERN DIGITAL under the trademark POLARIS. The screen 20 can provide alignment information to the surgeon including the angle between the femur and tibia mechanical axis. For this example, the system is an optical localizer; however, all other localizer like magnetic, ultrasound, accelerometer, etc. could be used under this innovation.

In order to compute the first bone mechanical axis and the second bone mechanical axis and to determine the alignment, joint centers must be acquired and estimated by the system. The proximal joint center can be measured following the FIG. 3. A 3D referential 21 associated to the tracker is fixed on the epiphysis of the tibia. The proximal joint center 4 can be found by making a circumduction motion 22 of the leg around the hip with a fixed relative position between the mechanical axis of the tibia 12, and the mechanical axis of the femur 11. For this example, the relative position is the full extension in order to have the needed information of the alignment for the surgeon, but other relative position (for instance in flexion) can be performed under this invention. The mechanical axis 11 and 12 must not move during the acquisition. 3D positions and orientations of the referential 21 are recorded by the system during the motion. Then, the computer uses a specific known algorithm with these recorded referentials such as for instance the center of rotation or the least moving point, etc. to determine the proximal center 4. The middle 3 and the distal joint 6 centers can be determined using the method described in the FIG. 4. A digitizer 23 composed of markers and a pointer 24, commercially available with the localizer system, such as, for instance, that marketed by the company NORTHERN DIGITAL, allows the system to obtain the 3D position of the pointer in the space. The system can obtain the middle joint center by acquiring with the digitizer the medial 25 and lateral 26 point of the tibia plateau and by computing the middle of these two points. The distal point can be determined in the same way: the medial 27 and the lateral 28 point of the ankle are acquired; the distal joint center is the middle of these two points. All other existing morphological or functional methods allowing the system to determine the joint centers can be used under this invention.

The FIG. 5 shows the overall surgical procedure with successive specific and innovative steps for the realignment of the limb. The surgeon must first fix the trackers on the future segments, epiphysis and diaphysis of the tibia (step 29). The surgeon makes a circumduction motion of the leg around the hip (step 31) while keeping fix the relative position of the femur and the tibia (step 30). The system, after recording the 3D positions and orientations of the epiphysis tracker, can estimate the proximal center in the epiphysis referential. The middle joint center (step 32) and the distal joint center (step 33) are then acquired with a digitizer with respect to, respectively, the epiphysis and the diaphysis referential. Once all acquisitions are performed, the surgeon can then perform the osteotomy, i.e. the cut and make an osseous hinge (step 34). The good alignment of the limb can be achieved by rotating the two segments around the hinge (step 35). The surgeon can visualize directly the result of the alignment with the navigation station (step 36) and modify, if required, the rotation between the two segments until reaching the desired alignment. The surgeon can then place the osteosynthesis implant to maintain the two bony segments in this configuration. This description was made for the lower limb but this innovation can also be used for all other limb;

The main advantages of the invention are to save time for the fixation of trackers and for the measurement of the alignment, and avoid more invasiveness for the patient.

1. Method of alignment of a first bone, with a first and a second end, in relation with a second bone, with a first and a second end, both bones being in a same limb, the first end of said first bone being a part of a distal joint for the first bone, the second end of said second bone being a part of a proximal joint for the second bone, said second end of the first bone and said first end of the second bone constituting a middle joint between both bones, optionally an articulation, wherein this first bone will be partially cut, in a transversal way, into two segments to form a hinge and wherein a tracker has been attached beforehand to each of two said bone segments, wherein said method comprises:
placing the two bones in a fixed relative position;
pivoting the two bones around said proximal joint and acquiring a point representing a center of rotation of said proximal joint;
acquiring a point representing a center of said middle joint, which is the middle of a medial anatomical point and a lateral anatomical point of said middle joint;
acquiring a point representing a center of said distal joint, which is the middle of the medial anatomical point and the lateral anatomical point of the said distal joint;
cutting partially said first bone into two segments to obtain said hinge;
pivoting the two segments of the said first bone around said hinge until reaching a desired alignment between said second bone and said two segments of the first bone, while acquiring relative positions of said three centers and;
visualizing alignment of the three joint points in relation to rotation of one segment with respect to the other one around said hinge.

2. The method of claim 1, wherein the proximal joint center of the second bone is obtained by performing a pivoting motion around the said proximal joint, recording said motion with a navigation system, and computing a center of rotation, following an optimization criterion.

3. The method of claim 1, wherein the middle joint center is computed by acquiring two points with a digitizer visible by said navigation system.

4. The method of claim 1, wherein the distal joint center is computed by acquiring two points with said digitizer visible by said navigation system.

5. System of alignment of a first bone, with a first and a second end, in relation with a second bone, with a first and a second end, both bones being in a same limb, the first end of said first bone being a part of a distal joint for the first bone, the second end of said second bone being a part of a proximal joint for the second bone, said second end of the first bone and said first end of the second bone constituting a middle joint, optionally an articulation between both bones, said first bone being partially cut, in a transversal way, into two segments linked partially to form a hinge, wherein said system comprises:
a computer assisted surgery station, which includes a screen and a computer, said computer comprising a memory, in which is recorded a computer program, and a treatment unit adapted to execute said program;
two trackers adapted to be placed each one on one of the two segments of said first bone, a position and orientation of said trackers being detectable by said station, further wherein said computer program comprises instructions for implementing the following:
while each tracker having been fixed respectively to each of said segments, and while a rotation of both bones is made around said proximal end of said second bone, said two bones being maintained in a fixed relationship one to the other, acquiring a point representing a proximal end of said second bone, in a referential of one of said trackers,
acquiring a point which represents a center of said articulation, in a referential of one of said trackers;
acquiring a point which represents a distal end of said first bone, in a referential of one of said trackers;
displaying, on a screen of said station, of said points which represent a proximal end of said second bone, a center of said articulation and a distal end of said first bone, during a movement of said two segments of said first bone, relative to each other.

6. The system of claim 5, wherein said screen and/or said computer is carried by one of said trackers.

7. The system of claim 5, wherein said trackers are optic.

8. The system of claim 5, wherein said trackers are magnetic.
